(19) United States
(12) Patent Application Publication
(10) Pub. No.: US 2017/0028146 A1
(43) Pub. Date: Feb. 2, 2017

(54) ENDOTRACHEAL TUBE SYSTEM

(71) Applicant: Jasphine NANDIGAMA, East Meadow, NY (US)

(72) Inventor: Jasphine NANDIGAMA, East Meadow, NY (US)

(21) Appl. No.: 14/813,560

(22) Filed: Jul. 30, 2015

(51) Int. Cl. A61M 16/00; A61M 16/04

(52) U.S. Cl. CPC A61M 16/0051; A61M 16/0003; A61M 16/04; A61M 2016/0027; A61M 2205/18; A61M 2205/3368; A61M 2205/3306

(57) ABSTRACT

An endotracheal tube (ETT) system is provided. The ETT system includes a monitoring device and an ETT including at least one sensor disposed thereon. The at least one sensor is in operable communication with the monitoring device for detecting removal of the ETT from a patient.

### BACKGROUND

Field of the Disclosure

The present disclosure relates to an endotracheal tube (ETT) system, and more particularly, to an ETT system including an ETT including a sensor at a proximal portion thereof and a monitoring device.

Description of the Related Art

ETTs are used for airway management and patency for various medical situations. These particular patient treatment times usually are either chosen voluntarily (pending a major surgery) or in emergency conditions (e.g., an accident requiring establishment of an airway, or a code blue, where patient is in a compromised situation and in danger of losing his/her life). It is in these difficult conditions that a medical professional intubates the patient.

Conventional ETT systems can include an ETT that is connected to or in operable communication with a monitoring unit that is configured to detect unintended extubation of the endotracheal tube from a patient, i.e., when a patient removes the tube. In such systems, the monitoring unit is configured to detect a change of a rate of airflow through the endotracheal tube, and, as a result of extubation, provide an alert to a medical professional indicating that the endotracheal tube has been removed.

While such systems are satisfactory for detecting unintended extubation of the endotracheal tube from a patient, these systems require the medial professional to re-intubate the patient, which can be time consuming for the medical professional, traumatic to the patient, increase artificial ventilation time, and requires excessive use of restrictive methods, e.g., restraints.

### SUMMARY

As can be appreciated, an ETT system including an ETT including a sensor at a proximal end thereof and a monitoring device may prove useful in the medical field.

Embodiments of the present disclosure are described in detail with reference to the drawing figures wherein like reference numerals identify similar or identical elements. As used herein, the term “distal” refers to the portion that is being described which is further from a user (qualified medical professional), while the term “proximal” refers to the portion that is being described which is closer to a user.

An aspect of the present disclosure provides an ETT system. The ETT system includes a monitoring device and an ETT including at least one sensor disposed thereon, the at least one sensor in operable communication with the monitoring device for detecting removal of the ETT from a patient.

An aspect of the present disclosure provides an ETT that is configured for use with a monitoring device. The ETT includes at least one sensor that is in operable communication with the monitoring device for detecting removal of the ETT from a patient.

An aspect of the present disclosure provides an ETT system. The ETT system includes a monitoring device and an ETT including at least one sensor disposed thereon, the at least one sensor in operable communication with the monitoring device for detecting when a patient grasps a proximal end of the ETT in an attempt to extubate the ETT.

An aspect of the present disclosure provides an invasive system. The invasive system includes a monitoring device and an invasive device including at least one sensor disposed thereon, the at least one sensor in operable communication with the monitoring device for detecting when a patient grasps a proximal end of the invasive device in an attempt to remove the invasive device.

### BRIEF DESCRIPTION OF THE DRAWING

Various embodiments of the present disclosure are described hereinbelow with references to the drawings, wherein:

FIG. 1 is a perspective view of an ETT system including an ETT and a monitoring device, in accordance with an embodiment of the present disclosure; and

FIGS. 2A and 2B are cross-sectional views of an ETT, in accordance with an embodiment of the present disclosure.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

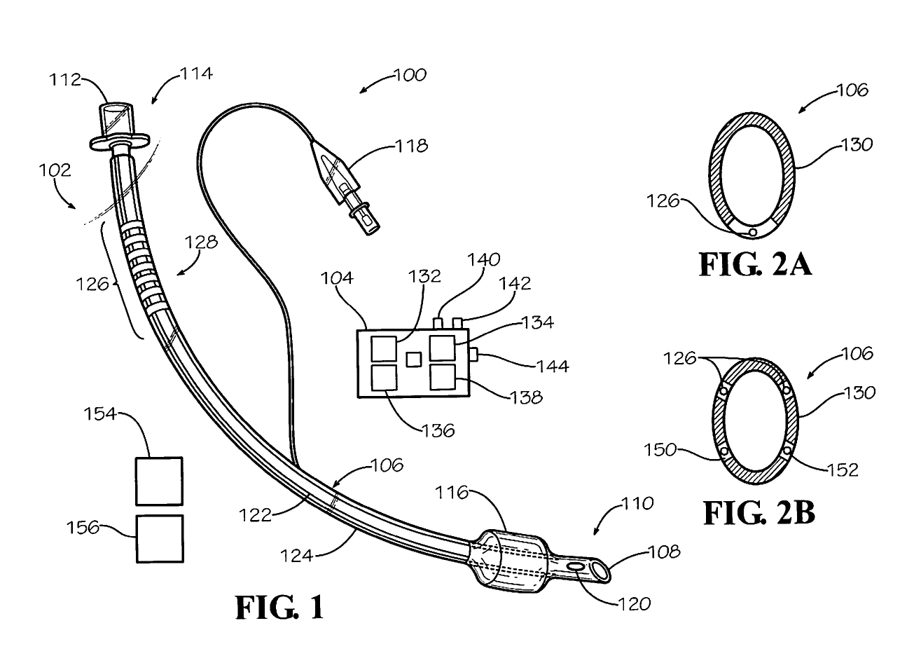

FIG. 1 is a perspective view of an ETT system 100, in accordance with an embodiment of the present disclosure. The ETT system 100 includes an ETT 102 and a monitoring device 104

The ETT 102 is comprised of a flexible, plastic tubular member 106 with an opening 108 at the distal end 110 and an opening 112 at the proximal end 114. A ventilator or other breathing apparatus is connected to the proximal end opening 112.

Adjacent to the distal end 110 is a cuff 116, e.g., an inflatable balloon, that, when inflated, retains the ETT 102 in position in the airway and closes the airway outside of the ETT 102. A pilot balloon 118 is used to inflate the cuff 116.

An opening 120 in the side of the tubular member 106 adjacent to the distal end 110, also called a Murphy Eye, prevents respiratory obstruction in the event the distal opening 108 becomes plugged.

In order for the ETT to be visible in an x-ray, it is known in the art to incorporate a radiopaque stripe 122 longitudinally in the tubular member 106 along at least a distal portion 124 of the tubular member 106 and end at or near the distal end 110.

One or more pressure sensors 126 (a plurality of pressure sensors 126 are shown FIG. 1) are disposed at a proximal portion 128 of the ETT. The one or more pressure sensors 126 are arranged along the proximal portion 128 so as to detect when a patient grasps the proximal portion 128.

The one or more pressure sensors 126 can be disposed along an exterior surface of the tubular member 106 and affixed thereto via one or more suitable affixation methods and/or devices, e.g., adhesive, transparent tape or film, etc. Alternatively, the one or more pressure sensors 126 can be embedded within an interior wall portion 130 of the tubular member 106, see FIGS. 2A and 2B, for example.

The one or more pressure sensors 126 detect when a user grasps the proximal portion 128 of the tubular member 106. More particularly, the one or more pressure sensors 126 are configured to detect when a pressure applied to the proximal portion 128 exceeds a predetermined value. The predetermined value can be set in accordance with a manufacturers or users demands.

The one or more pressure sensors 126 are in wireless communication with the monitoring device 104. One or more various short range wireless protocols including, but not limited to, Bluetooth®, Wifi®, Zigbee®, NFC, etc. may be used for providing a wireless communication interface between the one or more sensors 126 and the monitoring device 104.

The monitoring device 104 includes a microcontroller 132 which controls the overall functions of the monitoring device 104. An antenna 134, which is controlled by the microcontroller 132, receives signaling from the at least one or more pressure sensors 126. The signaling may include data such as pressure data that is detected by the one or more sensors 126.

The antenna 134 transmits the received data to one more demodulators 136 which demodulate the signaling received from the antenna 134. The demodulated signal is transmitted to the microcontroller 132 which determines if the detected pressure exceeds a predetermined pressure value. If the pressure exceeds the predetermined pressure value, the microcontroller 132 transmits a control signal to a speaker 138 of the monitoring device 104 requesting that the speaker 138 output one or more audio signals, e.g., an alarm signal.

The monitoring device 104 also includes an on/off button/switch 140 and a volume control device 142, e.g., a knob, which are in communication with the microcontroller 132 for turning on/off the monitoring device 104 and adjusting a volume of the output of speaker 138.

One or more visual indicators 144, e.g., light emitting diodes, may also be provided on the monitoring device 104.

In use, a medical professional, e.g., a doctor or qualified medical professional, intubates a patient. As is customary practice, the proximal portion 128 of the ETT 102 remains outside of the patient for connecting the proximal end opening 112 to a ventilator or other breathing apparatus.

The medical professional turns on the monitoring device 104. If the patient grasps the proximal portion 128 in an attempt to pull out the ETT 102, the one or more pressure sensors 126 disposed at the proximal portion 128 detects a pressure exerted at the proximal portion 128. When the pressure is detected, the one or more pressure sensors 126 transmit signaling which is detected by the antenna 134 of the monitoring device 104.

The antenna 134 transmits the received pressure data to the microcontroller 132 which analyzes the pressure data. If it is determined that the detected pressure exceeds a predetermined value, the microcontroller 132 transmits a control signal to the speaker 138 of the monitoring device 104 requesting that the speaker 138 output an alarm signal to alert the medical professional that the patient is attempting to remove the ETT. The microcontroller 132 can also control the monitoring device 104 to output a visual indication, e.g., light the LED, to the medical professional.

The ETT system 100 including the ETT 102 and monitoring device 104 overcome the aforementioned drawbacks that are associated with conventional ETT systems. That is, unlike conventional ETT systems that are configured to alert a medical professional only after a patient has extubated the ETT, the ETT system 100 is configured to alert a medical professional prior to the patient extubating the ETT. Accordingly, the need for a medical professional to re-intubate the patient, which would demand more of the medical professional's time and which would cause additional pain to the patient, would be eliminated.

From the foregoing and with reference to the various figure drawings, those skilled in the art will appreciate that certain modifications can also be made to the present disclosure without departing from the scope of the same. For example, while the one or more sensors 126 have been described herein in terms of pressure sensors, the present disclosure is not so limited. For example, other sensors can be disposed at the proximal portion 128 of the ETT 102. For example, light sensors 150 and/or temperature sensors 152 can be used in conjunction with or in place of the one or more pressure sensors 126. For illustrative purposes, in FIG. 2B, the proximal portion 128 is shown including two pressure sensors 126, a light sensor 150 and a temperature sensor 152.

In the embodiment illustrated in FIG. 2B, the light sensor 150 and temperature sensor 152 are configured to transmit signaling that is detected by the antenna 134. The detected signaling can be processed in a manner as described above with respect to the one or more pressure sensors 126. As can be appreciated, one or more modifications to the monitoring device 104 may have to be made in order to accommodate the light sensors 150 and temperature sensors 152. For example, the microcontroller 132 is configured to determine if light value falls below a predetermined value or if a temperature value exceeds and/or falls below a predetermined value.

While the one or more sensors 126, 150, and 152 have been described herein as communicating with the monitoring device 104 via a wireless communication scheme, other communication schemes may be used. For example, a wired communication scheme may be used instead of a wireless communication scheme. As can be appreciated, one or more wires may be connected to the one or more pressure sensors 126 and connected to the monitoring device 104; the one or more wires can be directly or indirectly connected to the monitoring device 104.

Moreover, the monitoring device 104 can be positioned in the same room as the patient, or the monitoring device can be positioned at a location that is remote, e.g., a nurse's station, relative to the patient; various factors will play a role in determining where the monitoring device 104 is located with respect to the patient. For example, a manufacturer's or a user's preference, a communication scheme used between the one or more sensors and the monitoring device, are just but a few.

The ETT 102 and the monitoring device 104 of the ETT system 100 can be sold separately or together as a kit.

While the one or more sensors 126, 150, and 152 have been described herein as being used with a system including the ETT 102, it is within purview of the present disclosure that the sensors can also be provided on other invasive lines, tubes, or devices, e.g., a chest tube 154, nasogastric tube 156, which are shown schematically in FIG. 1. As can be appreciated, one or more modifications may have to be made to a system that includes such devices in order for that system to function as described herein with respect to the system 100.

At least a part of the monitoring device 104 (e.g., modules or the functions) or methods (e.g., operations) described above may be implemented with, for example, instructions stored in a non-transitory computer-readable storage media which has a program module. When the instructions are executed by a processor (e.g., the microcontroller 132), one or more processors may perform functions corresponding to the instructions. The non-transitory computer-readable storage media may be, for example, a memory 146 (FIG. 1), which is used to store various data that can be read from the other modules of the monitoring device 104.

The non-transitory computer-readable storage media may include a hard disc, a floppy disk, magnetic media (e.g., a magnetic tape), optical media (e.g., a compact disc read only memory (CD-ROM), a digital versatile disc (DVD), and magneto-optical media (e.g., a floptical disk)), a hardware device (e.g., a ROM, a random access memory (RAM), or a flash memory, and the like), and the like. Also, the program instructions may include not only mechanical codes compiled by a compiler (not shown) but also high-level language codes which may be executed by a computer using an interpreter and the like. The above-mentioned hardware device may be configured to operate as one or more software modules to perform operations according to various embodiments of the present invention, and vice versa.

The non-transitory computer-readable storage media may include instructions which may be executed by at least one processor. The instructions may be set to determine whether a predetermined pressure value has been exceeded or whether one or more of the other aforementioned values, e.g., a temperature value and/or a light value, exceeds and/or falls below a respective predetermined value. In the event that it is determined a predetermined value has been met, the instructions, when set, may instruct the speaker to output one or more of the aforementioned alerts.

The non-transitory computer readable medium may be downloaded on one or more wireless devices including, but not limited to, smart phones, tablets and other suitable wireless devices. For example, the instructions can be downloaded onto a smart phone. In this instance, the smart phone can communicate with the one more sensors described above and can perform the functions of the monitoring device 104. For example, the instructions can be set to synchronize the one or more sensors with the smart phone such that the smart phone can perform the functions of the monitoring device described above. For example, the smart phone can receive the signaling from the one or more sensors and the processor of the smart phone can be configured to determine if a predetermined value has been met. Upon determination of the predetermined value being met, the instructions may instruct the speaker of the smart phone to output one or more of the aforementioned alerts, e.g., a visual alert on a screen of the smart phone, an audio alert output from a speaker of the smart phone, or a tactile feedback (vibration) from the smart phone.

Modules or program modules according to various embodiments of the present disclosure may include at least one or more of the above-mentioned components, some of the above-mentioned components may be omitted, or other additional components may be further included. Operations executed by modules, program modules, or other components may be executed by a successive method, a parallel method, a repeated method, or a heuristic method. Also, some operations may be executed in a different order or may be omitted, and other operations may be added.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An endotracheal tube (ETT) system, comprising:
a monitoring device; and
an ETT including at least one sensor disposed thereon, the at least one sensor in operable communication with the monitoring device for detecting removal of the ETT from a patient.

2. The ETT system according to claim 1, wherein the monitoring device is in at least one of wireless communication and wired communication with the at least one sensor.

3. The ETT system according to claim 1, wherein the at least one sensor is one of a pressure sensor, a light sensor, and a temperature sensor.

4. The ETT system according to claim 1, wherein the at least one sensor is disposed at a proximal portion of the ETT.

5. The ETT system according to claim 1, wherein the at least one sensor is a plurality of sensors.

6. The ETT system according to claim 1, wherein the at least one sensor is disposed on an exterior surface of the ETT.

7. The ETT system according to claim 1, wherein the at least one sensor is disposed within an interior wall portion of the ETT.

8. The ETT system according to claim 1, wherein the monitoring device is configured to alert, via one of an audio and a visual signal, a medical professional when a patient attempts to remove the ETT tube.

9. The ETT system according to claim 1, wherein the monitoring device includes at least one of an on/off switch and a volume control.

10. An endotracheal tube (ETT) configured for use with a monitoring device, the ETT comprising:
at least one sensor that is in operable communication with the monitoring device for detecting removal of the ETT from a patient.

11. The ETT according to claim 10, wherein the at least one sensor is in at least one of wireless communication and wired communication with the monitoring device.

12. The ETT according to claim 10, wherein the at least one sensor is one of a pressure sensor, a light sensor, and a temperature sensor.

13. The ETT according to claim 10, wherein the at least one sensor is disposed at a proximal end of the ETT.

14. The ETT according to claim 10, wherein the at least one sensor is a plurality of sensors.

15. The ETT according to claim 10, wherein the at least one sensor is disposed on an exterior surface of the ETT.

16. The ETT according to claim 1, wherein the at least one sensor is disposed within an interior wall portion of the ETT.

17. An endotracheal tube (ETT) system, comprising:
a monitoring device; and
an ETT including at least one sensor disposed thereon, the at least one sensor in operable communication with the monitoring device for detecting when a patient grasps a proximal end of the ETT in an attempt to extubate the ETT.

18. The ETT system according to claim 17, wherein the at least one sensor is one of a pressure sensor, a light sensor, and a temperature sensor.

19. The ETT system according to claim 17, wherein the at least one sensor is a plurality of sensors.

20. The ETT system according to claim 17, wherein the at least one sensor is disposed on one of an exterior surface of the ETT and within an interior wall portion of the ETT.

21. An invasive system, comprising:
a monitoring device; and
an invasive device including at least one sensor disposed thereon, the at least one sensor in operable communication with the monitoring device for detecting when a patient grasps a proximal end of the invasive device in an attempt to remove the invasive device.

22. The invasive line tube system according to claim 21, wherein the invasive device is selected from the group consisting of an endotracheal tube (ETT), a chest tube, and nasogastric tube.
